(19) United States
(12) Patent Application Publication
(10) Pub. No.: US 2026/0034343 A1
(43) Pub. Date: Feb. 5, 2026

(54) OUTSIDE AORTIC COUNTERPULSATION DEVICE WITH EXTRACORPOREAL RESPIRATORY CHAMBER

(71) Applicant: Taizhou MYDsn Medical Technology Co., Ltd, Taizhou (CN)

(72) Inventors: Xuerui TAN, Beijing (CN); Haitao JIA, Tianjin (CN); Guojun HE, Wuhan (CN)

(73) Assignee: Taizhou MYDsn Medical Technology Co., Ltd, Taizhou (CN)

(21) Appl. No.: 18/888,217

(22) Filed: Sep. 18, 2024

(30) Foreign Application Priority Data
Aug. 5, 2024 (CN) 202411065509.4

(51) Int. Cl. A61M 60/117

(52) U.S. Cl. CPC A61M 60/117

(57) ABSTRACT

An outside aortic counterpulsation device with an extracorporeal respiratory chamber includes an intracorporeal catheter and a respiratory chamber assembly. The respiratory chamber assembly is connected to a counterpulsation controller through a wire. A side wall of an inner end of the intracorporeal catheter is provided with a plurality of through holes, and an outer end of the intracorporeal catheter is hermetically connected to an extracorporeal balloon. The respiratory chamber assembly includes a housing and a piston driving mechanism and an elastic respiratory chamber membrane arranged inside the housing. A closed end of the elastic respiratory chamber membrane is connected to a piston, and an open end of the elastic respiratory chamber membrane wraps around the extracorporeal balloon and is hermetically connected to an outer side of a base of the extracorporeal balloon. The piston driving mechanism is configured to drive the elastic respiratory chamber membrane to expand or contract.

### CROSS REFERENCE TO THE RELATED APPLICATIONS

This application is based upon and claims priority to Chinese Patent Application No. 202411065509.4, filed on Aug. 5, 2024, the entire contents of which are incorporated herein by reference.

### TECHNICAL FIELD

The present disclosure relates to the technical field of medical devices, and in particular to an outside aortic counterpulsation device with an extracorporeal respiratory chamber.

### BACKGROUND

An intra aortic balloon pump (IABP) is a mechanical device designed based on the principle of counterpulsation to provide assistance to a failing left ventricle. It is commonly used for in-hospital rehabilitation after interventional cardiac surgery, heart attacks, or other major cardiac events. As the most commonly used auxiliary device in the perioperative period of percutaneous coronary intervention (PCI), IABP is particularly suitable for providing advanced life support to critically ill cardiology patients, effectively improving coronary blood supply and alleviating symptoms of low cardiac output and hypotension. The basic principle of IABP is to enable a balloon placed in the descending aorta to inflate during diastole and deflate during systole to achieve cardiac circulation assistance. During diastole, the balloon in the aorta is inflated to increase diastolic blood pressure, increase coronary blood flow, improve myocardial blood supply and oxygen supply, and increase blood perfusion to the brain, kidneys, and periphery at the same time. During systole, the balloon is rapidly deflated, creating a “cavitation effect” that reduces left ventricular afterload and lowers myocardial oxygen consumption.

In the interventional treatment of cardiovascular diseases, IABP is the most commonly used auxiliary device in the perioperative period of PCI. According to data from the National Cardiovascular Data Registry (NCDR) in the United States from 2009 to 2013, among patients using percutaneous mechanical circulatory support (pMCS), 89.3% of patients used IABP, while only 10.7% of patients used other assistive devices. Currently, all IABPs used in China are imported from abroad. The major global manufacturers of IABPs include Getinge Group, Teleflex, and Senko Medical instrument Mfg. Co., Ltd. (MERA). In 2020, Getinge Group's IABP share accounted for over 80%. According to the selection criteria of IABP balloons, the balloon size is determined based on body height. At present, Teleflex's Arrow IABP system includes catheters with diameters of 7 fr, 7.5 fr, and 8 fr, and balloons with capacities of 30 cc, 40 cc, and 50 cc.

Based on comparisons of the use of IABP, extracorporeal membrane oxygenation (ECMO), and artificial hearts, IABP has absolute advantages in terms of sheath size, bedside placement, operational difficulty, retention time, postoperative management requirements, and risk of hemolysis. The only drawback of IABP lies in the low cardiac output, which is only 0.5-1 L/min. Therefore, in terms of actual effectiveness in patients, IABP is significantly inferior to ECMO and artificial hearts. To address this significant deficiency of IABP, international and domestic doctors and engineers have tried many methods, but with little success, and there is still no effective solution to solve the problem of low cardiac output in IABP.

In view of the above, the present disclosure creatively proposes an outside aortic counterpulsation device with an extracorporeal respiratory chamber by fully utilizing the advantages of IABP. The present disclosure adopts an outside aortic counterpulsation method, which places a balloon-like device outside the body to maximize cardiac output, thereby solving the problem of low cardiac output in IABP.

### SUMMARY

A technical problem to be solved by the present disclosure is to provide an outside aortic counterpulsation device with an extracorporeal respiratory chamber to maximize cardiac output and thereby solve the problem of low cardiac output in an intra aortic balloon pump (IABP).

In order to address the above technical problem, the present disclosure provides an outside aortic counterpulsation device with an extracorporeal respiratory chamber, including an intracorporeal catheter extending into a descending aorta and a respiratory chamber assembly located outside a body, where one end of the respiratory chamber assembly is connected to an outer end of the intracorporeal catheter, and the other end of the respiratory chamber assembly is connected to a counterpulsation controller;
a side wall of an inner end of the intracorporeal catheter is provided with a plurality of through holes, and the outer end of the intracorporeal catheter is hermetically connected to an extracorporeal balloon; and
the respiratory chamber assembly includes a housing and a piston driving mechanism and an elastic respiratory chamber membrane arranged inside the housing; a closed end of the elastic respiratory chamber membrane is fixedly connected to a piston of the piston driving mechanism, and an open end of the elastic respiratory chamber membrane wraps around the extracorporeal balloon and is hermetically connected to an outer side of a base of the extracorporeal balloon; a respiratory chamber is formed between the elastic respiratory chamber membrane and the extracorporeal balloon; the piston driving mechanism is configured to drive the elastic respiratory chamber membrane to expand or contract; when the elastic respiratory chamber membrane expands, a vacuum is formed inside the respiratory chamber, causing the extracorporeal balloon to be blown in an opposite direction, thereby drawing blood from the descending aorta into the extracorporeal balloon; and when the elastic respiratory chamber membrane contracts, an internal space of the respiratory chamber is squeezed, causing the blood in the extracorporeal balloon to be perfused into a body, thereby achieving outside aortic blood counterpulsation.

As a further improvement, a lumen diameter of the intracorporeal catheter is 2.2-9 mm; a hole setting section of the intracorporeal catheter has a length of 320-520 mm; the through hole has a diameter of 1-4 mm; and any two adjacent through holes have a spacing of 1-10 mm.

As a further improvement, a diaphragm is provided in the intracorporeal catheter and configured to divide an internal space of the catheter into a blood drawing chamber and a perfusion chamber; and one end of the diaphragm is located at a hole setting starting position of the intracorporeal catheter, and the other end of the diaphragm is located at the base of the extracorporeal balloon.

As a further improvement, two sides of the diaphragm are respectively adhered to an inner side wall of the intracorporeal catheter; and the two ends of the diaphragm are inclined, such that an inner end of the blood drawing chamber and an outer end of the perfusion chamber form a horn mouth-shaped structure.

As a further improvement, the diaphragm is an elastic membrane.

As a further improvement, the intracorporeal catheter is made of a polymer material of polytetrafluoroethylene (PTFE), polyurethane (PU), Pebax or polyethylene (PE) through a blow molding or extrusion process; the extracorporeal balloon and the elastic respiratory chamber membrane each are made of elastic silicone rubber or PU; and the membrane is made of a medical polymer material of elastic silicone rubber, PU, PTFE, PU, Pebax or PE, and the membrane has a thickness of 0.5-1.5 mm.

As a further improvement, each of the two ends of the diaphragm is provided with a one-way valve; the one-way valve includes a valve element and a rotating shaft; the rotating shaft is fixed to the end of the diaphragm; one end of the valve element is rotatably connected to the rotating shaft, and the other end of the valve element matches an inner side wall structure of the intracorporeal catheter; the one end of the valve element connected to the rotating shaft is further provided with a limit element for limiting a rotation angle of the valve element; and valve elements at the two ends of the diaphragm are arranged in opposite directions to alternately achieve the opening and closing of the blood drawing chamber and the perfusion chamber.

As a further improvement, the valve element is made of a hard polymer material, a metal material, or a ceramic material.

As a further improvement, a pressure sensor is provided at an outer side of the inner end of the intracorporeal catheter.

As a further improvement, the counterpulsation controller is an IABP system controller for providing electrical energy and a control signal to the respiratory chamber assembly.

With such a design, the present disclosure has at least the following advantages:
1. In the present disclosure, the intracorporeal catheter of the outside aortic counterpulsation device includes an outer end side provided with an extracorporeal balloon and an inner end side provided with a plurality of through holes. When in use, the intracorporeal catheter and the blood vessel form a closed space, and the extracorporeal balloon relies on the respiratory chamber assembly located outside the body to expand or contract, achieving extracorporeal counterpulsation of blood in the descending aorta. Through this structure, the volume of the extracorporeal balloon is greatly increased, with a maximum volume of up to 200 ml, ultimately achieving a cardiac output of over 5 L/min, thereby meeting the patient's systemic blood supply needs and greatly improving the patient's blood supply condition. In this structure, the respiratory chamber is far away from the aorta, and even in case of high-risk events such as respiratory chamber leakage, it can be promptly blocked to prevent adverse effects on the patient and improve safety.
2. The diaphragm in the intracorporeal catheter provides a dual-channel structure in the intracorporeal catheter, with one side used for blood drawing and the other side used for perfusion. The two channels do not affect each other, avoiding the problems of blood collision and severe turbulence that exist in single-channel structures.
3. The elastic membrane and the two horn mouth-shaped ends of the elastic membrane effectively ensure one-way blood flow and maximize the one-way cross-section during blood drawing and perfusion, thereby greatly reducing external power and effectively achieving one-way blood flow.
4. The end of the diaphragm is provided with the one-way valve, which effectively ensure the one-way blood flow in the blood drawing chamber and the perfusion chamber, greatly reducing external power and facilitating blood flow.
5. In the present disclosure, the outside aortic counterpulsation device is driven by a motor. The design eliminates various defects of the air pump drive, such as leakage, compressibility of gas, and unclean gas, and can provide greater power, ensuring a larger cardiac output.

### BRIEF DESCRIPTION OF THE DRAWINGS

The above is only an overview of the technical solutions of the present disclosure. In order to understand the technical means of the present disclosure more clearly, the present disclosure will be further described in detail below with reference to the drawings and specific embodiments.

FIG. 1 is a structural diagram of an outside aortic counterpulsation device used in a body according to the present disclosure;

FIG. 2 is a structural diagram of an intracorporeal catheter of the outside aortic counterpulsation device according to the present disclosure;

FIG. 3 is a structural diagram of a respiratory chamber assembly of the outside aortic counterpulsation device when an extracorporeal balloon expands according to the present disclosure;

FIG. 4 is a structural diagram of the respiratory chamber assembly of the outside aortic counterpulsation device when the extracorporeal balloon contracts according to the present disclosure;

FIG. 5 is a structural diagram of a diaphragm setting section of the intracorporeal catheter of the outside aortic counterpulsation device according to the present disclosure;

FIG. 6 is a sectional view of the diaphragm setting section of the intracorporeal catheter of the outside aortic counterpulsation device when a blood drawing chamber expands according to the present disclosure;

FIG. 7 is a sectional view of the diaphragm setting section of the intracorporeal catheter of the outside aortic counterpulsation device when a perfusion chamber expands according to the present disclosure;

FIG. 8 is a structural diagram of a one-way valve at an end of the diaphragm of the outside aortic counterpulsation device according to the present disclosure;

FIG. 9 is a structural diagram of a one-way valve opened at a side of the diaphragm of the outside aortic counterpulsation device according to the present disclosure; and

FIG. 10 is a structural diagram of a one-way valve closed at a side of the diaphragm of the outside aortic counterpulsation device according to the present disclosure.

### DETAILED DESCRIPTION OF THE EMBODIMENTS

Exemplary embodiments of the present disclosure will be described in detail below with reference to the drawings. Although the drawings show exemplary embodiments of the present disclosure, it should be understood that the present disclosure may be implemented in various forms and should not be limited to the embodiments set forth herein. The embodiments are provided to provide a more thorough understanding of the present disclosure, and the scope of the present disclosure can be fully conveyed to those skilled in the art.

Finally, it should be noted that orientation or position relationships indicated by terms such as “left”, “right”, “inner”, and “outer” are based on the orientation or position relationships shown in the drawings. These terms are just used to facilitate the description of the present disclosure and simplify the description, but not to indicate or imply that the mentioned device or elements must have a specific orientation and must be established and operated in a specific orientation, and thus, these terms cannot be understood as a limitation to the present disclosure.

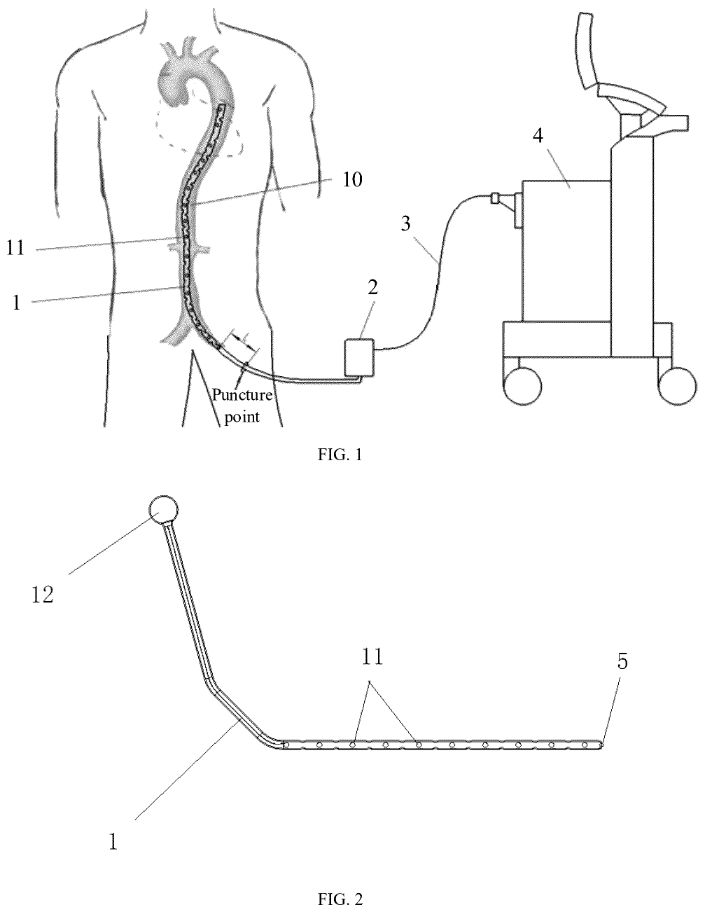

As shown in FIG. 1, an embodiment provides an outside aortic counterpulsation device with an extracorporeal respiratory chamber, including intracorporeal catheter 1 and respiratory chamber assembly 2. One end of the intracorporeal catheter 1 is configured to extend into descending aorta 10 inside a body. The respiratory chamber assembly 2 is located outside the body. One end of the respiratory chamber assembly 2 is connected to an outer end of the intracorporeal catheter 1, and the other end of the respiratory chamber assembly 2 is connected to counterpulsation controller 4 through wire 3.

As shown in FIG. 2, a side wall of an inner end of the intracorporeal catheter 1 is provided with a plurality of through holes 11, and an outer end of the intracorporeal catheter 1 is closed and connected to extracorporeal balloon 12. When the intracorporeal catheter 1 enters the body, the intracorporeal catheter 1 forms a closed space with a blood vessel inside the body, and blood flows through a pathway formed by the closed space.

Specifically, an overall length of the intracorporeal catheter 1 is 500-1,000 mm, preferably 600, 750, and 900 mm to accommodate patients of different heights. An outer diameter of the intracorporeal catheter 1 is minimally 7 F and maximally 24 F, which means a lumen diameter of 2.2-9 mm. The principle of setting the diameter is as follows. The diameter of the lumen of the human aorta is generally around 10 mm, so the outer diameter of the catheter must be smaller than the inner diameter of the blood vessel, while leaving a certain gap. A one-way gap of 0.5 mm is necessary, so the maximum outer diameter of the catheter cannot exceed 9 mm, otherwise it will damage the inner wall of the blood vessel. Of course, the catheter cannot be too thin. If the catheter is too thin, both drawing blood outward and infusing it inward will be very laborious, the load will be very large, and the hysteresis effect of the liquid will become very obvious. Meanwhile, during the process of vigorous drawing, a vacuum phenomenon may occur, which can damage blood cells and may also cause gas embolism, endangering the patient's life safety.

In addition, the through holes 11 of the intracorporeal catheter 1 are located in a hole setting section with a length of 320-520 mm. The plurality of through holes 11 are provided to reduce the load during blood drawing and perfusion, making it easy to achieve blood flow. The through holes 11 have a diameter of 1-4 mm, and are evenly distributed around a catheter wall. The distribution pattern is not limited and can be a spiral distribution pattern or an even circle-like distribution pattern. In an optimal pattern, any two through holes are equally spaced, with a spacing of 1-10 mm. If the spacing is too small, it will damage the overall stiffness of the catheter. If the spacing is too large, it will increase the drawing load. The hole size is also based on the same principle. If the through hole is too small, it will increase the drawing load and make it more difficult for drawing. If the through hole is too large, it will damage the overall stiffness and make the catheter prone to damage. It should be noted that the starting position of the through holes on the catheter wall should not be too close to a puncture point for an entry into the body, otherwise it may lead to bleeding. A preferred range for a distance L between the starting section of the through holes and the puncture point is 20-80 mm. If the distance is too short, there may be bleeding during the drawing and perfusion processes due to excessive pressure. If the distance is too large, a blind area without holes will be too large, resulting in an increase in drawing load and difficulty in drawing. An optimal range of L is 30-50 mm.

In this embodiment, pressure sensor 5 is provided at an outer side of the inner end of the intracorporeal catheter 1 for performing real-time monitoring of a blood pressure and transmitting a signal to the counterpulsation controller 4, forming a trigger mode for control.

The application principle of the outside aortic counterpulsation device is as follows. The intracorporeal catheter 1 is inserted into the aorta through puncture surgery, with its end located in the descending aorta 10. The intracorporeal catheter at most reaches the aortic arch, but does not enter the aortic arch. Ideally, the intracorporeal catheter should be approximately 100 mm away from the bend of the aortic arch. The surgical puncture site can be the femoral artery, radial artery, or carotid artery. After the puncture of the intracorporeal catheter 1 is completed, blood from the descending aorta 10 in the body is guided through the through holes 11 into the extracorporeal balloon 12 under the control of the respiratory chamber assembly 2 and the counterpulsation controller 4, and perfused back into the descending aorta 10 in the body to achieve outside aortic blood counterpulsation.

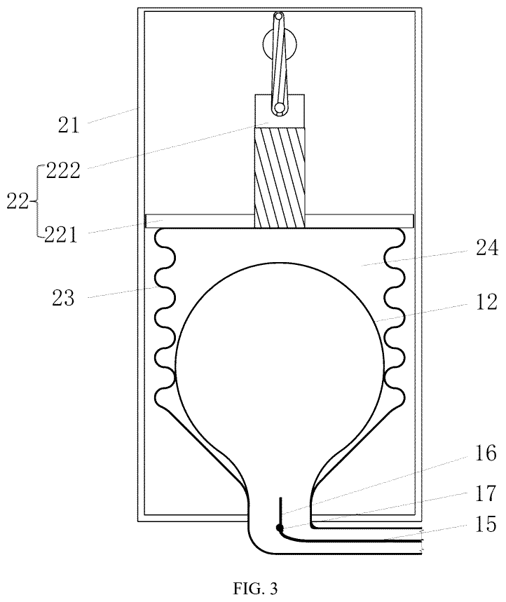

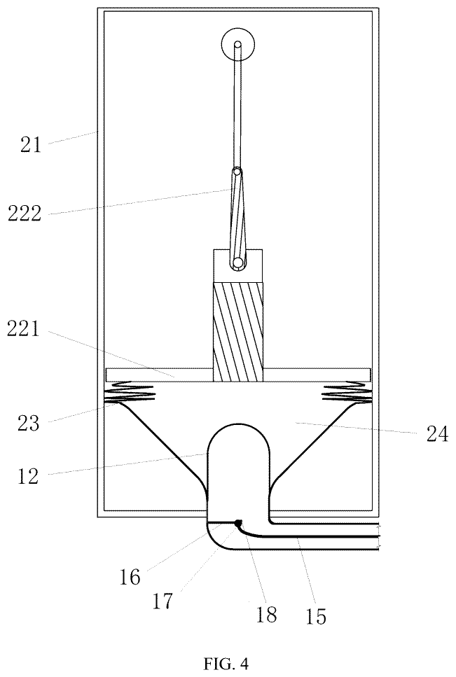

As shown in FIGS. 3 and 4, in this embodiment, the respiratory chamber assembly 2 includes housing 21 and piston driving mechanism 22 and elastic respiratory chamber membrane 23 arranged inside the housing 21. The housing 21 is configured to accommodate other components of the respiratory chamber assembly 2 and provide a rigid environment, allowing all work to occur internally without affecting the external environment. The housing 21 can be designed in the form of a backpack or shoulder bag for easy carrying by patients during activities.

The piston driving mechanism 22 includes piston 221 and driver 222 that drives the piston to extend and contract. The driver 222 can be a combination of a motor and a crank slider, a motor and a linear bearing, a linear motor or a voice coil motor. In order to draw 200 ml of blood into the extracorporeal balloon 12 and smoothly perfuse it, a motor with a thrust of 100 kg is selected. The housing 21 can be pre-evacuated and filled with safe gases such as helium, air, nitrogen, or safe liquids such as physiological saline, injection water, purified water. In addition, there may be some noise generated during the operation of the driver, so a noise reduction device can also be provided in the housing 21.

A closed end of the elastic respiratory chamber membrane 23 is fixedly connected to the piston 221 of the piston driving mechanism 22. An open end of the elastic respiratory chamber membrane 23 wraps around the extracorporeal balloon 12 and is hermetically connected to an outer side of a base of the extracorporeal balloon 12, for example, by means of adhesive or threaded connection. Respiratory chamber 24 is formed between the elastic respiratory chamber membrane 23 and the extracorporeal balloon 12. The elastic respiratory chamber membrane 23 is a flexible elastic membrane that can expand and contract, and can be made of a highly elastic material such as silicone rubber and polyurethane (PU). The extracorporeal balloon 12 is driven by the piston driving mechanism 22, driving the elastic respiratory chamber membrane 23 to expand and contract repeatedly. When the elastic respiratory chamber membrane 23 expands, a vacuum is formed inside the respiratory chamber 24, causing the extracorporeal balloon 12 to be blown in the opposite direction, thereby drawing the blood from the descending aorta into the extracorporeal balloon 12, as shown in FIG. 3. When the elastic respiratory chamber membrane 23 contracts, the internal space of the respiratory chamber 24 is squeezed, which promotes blood perfusion back into the body through the extracorporeal balloon 12, as shown in FIG. 4, thereby achieving outside aortic blood counterpulsation flow.

In a preferred embodiment, as shown in FIG. 5, diaphragm 15 is provided in the intracorporeal catheter 1 and configured to divide the internal space of the catheter into blood drawing chamber 13 and perfusion chamber 14. One end of the diaphragm 15 is located at a hole setting position of the intracorporeal catheter 1, and the other end of the diaphragm 15 is located at the base of the extracorporeal balloon 12.

Specifically, two sides of the diaphragm 15 are respectively adhered to an inner side wall of the intracorporeal catheter 1. Two ends of the diaphragm 15 are inclined, such that an inner end of the blood drawing chamber 13 and an outer end of the perfusion chamber 14 form a horn mouth-shaped structure, as shown in FIG. 5, providing guidance for the flow of blood in the intracorporeal catheter and ensuring smooth blood inflow and outflow.

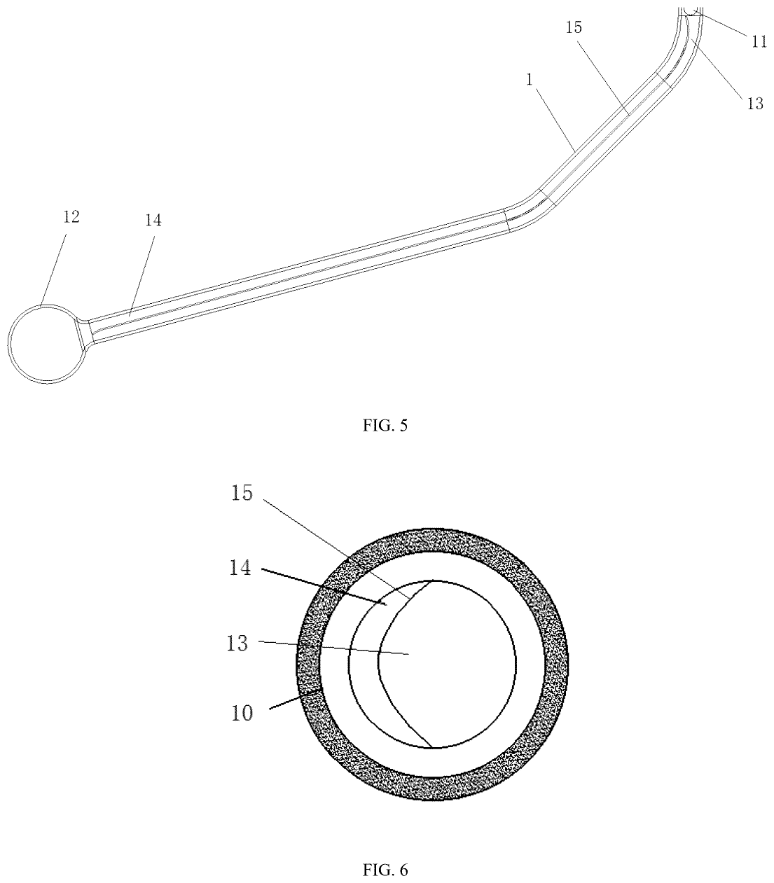

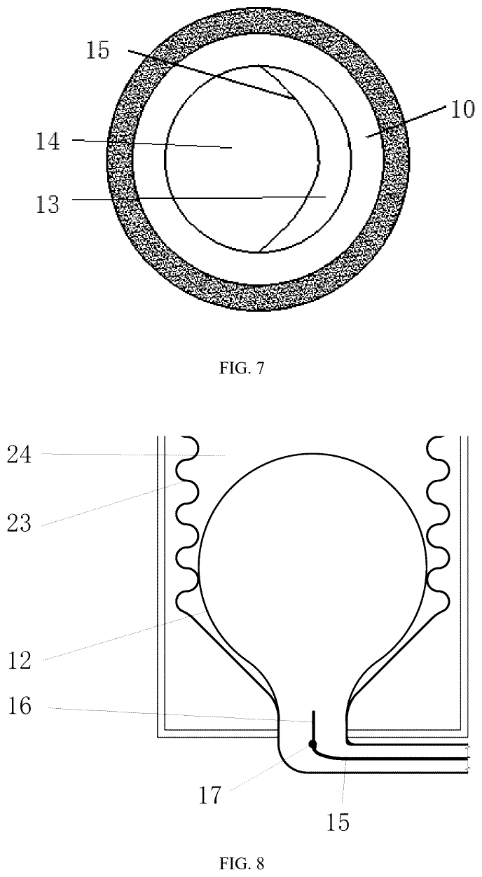

More preferably, the diaphragm 15 is an elastic membrane made of a medical polymer material such as elastic silicone rubber, PU, polytetrafluoroethylene (PTFE), PU, Pebax or polyethylene (PE). During the blood drawing process, when the elastic membrane deforms to the left, it presses against the perfusion chamber 14, causing the blood drawing chamber 13 to become larger and the perfusion chamber 14 to become smaller, as shown in FIG. 6. During the perfusion process, when the membrane deforms to the right, it presses against the blood drawing chamber 13, causing the perfusion chamber 14 to become larger and the blood drawing chamber 13 to become smaller, as shown in FIG. 7. The design achieves adaptive adjustment during drawing and perfusion processes. In the initial state, the membrane is centered and divides the lumen of the catheter equally, that is, the cross-sectional areas of the blood drawing chamber 13 and the perfusion chamber 14 are the same. The reasons for designing the dual-chamber structure are as follows. If a single-channel catheter is used, during the processes of blood drawing and perfusion, the blood in the lumen will remain in a repeated process of acceleration→deceleration→stop→reverse acceleration→reverse deceleration→stop. In this process, due to high frequency and fast speed, the liquid cannot be fed back quickly, resulting in a vacuum phenomenon and causing the liquid to collide, leading to severe turbulence. If dual channels are used, the blood is drawn in from one end and perfused back from the other end, avoiding mutual interference. Correspondingly, as the speed increases, vacuum and turbulence phenomena disappear, and the required external power is greatly reduced. Therefore, in the present disclosure, the dual-chamber structure of the catheter ensures unidirectional blood flow. Furthermore, if only one inelastic membrane is set up to divide one chamber into two chambers, the cross-section of each chamber will become very small, less than or equal to half of the original cross-section. This will increase the difficulty of drawing and perfusion. Therefore, in the present disclosure, the creative elastic membrane is designed with a horn mouth-shaped structure at each end, effectively ensuring one-way blood flow and maximizing the cross-sectional area of the two parts during operation, thereby greatly reducing external power and achieving one-way blood flow.

More specifically, the intracorporeal catheter 1 is made of a polymer material of PTFE, PU, Pebax or PE through a blow molding or extrusion process. The extracorporeal balloon 12 and the elastic respiratory chamber membrane 23 each are made of silicone rubber or PU.

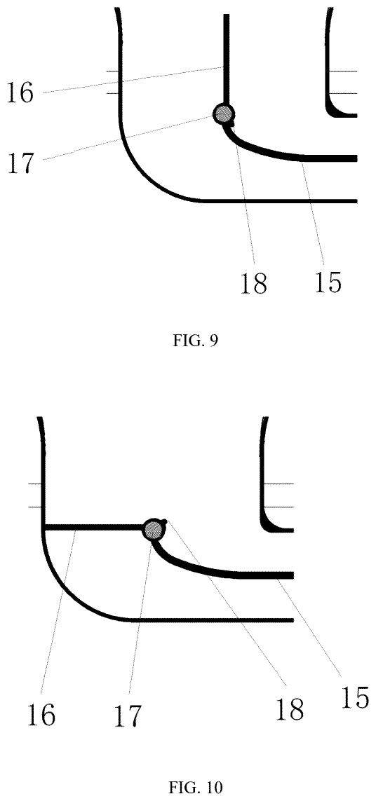

More preferably, as shown in FIGS. 8, 9, and 10, in this embodiment, each of the two ends of the diaphragm 15 is provided with one-way valve for one-way blood flow. The one-way valve includes valve element 16 and rotating shaft 17. The rotating shaft 17 is fixed to the end of the diaphragm 15. One end of the valve element 16 is rotatably connected to the rotating shaft 17, and the other end of the valve element 16 matches an inner side wall structure of the intracorporeal catheter 1. The one end of the valve element 16 connected to the rotating shaft 17 is further provided with a limit element 18 for limiting a rotation angle of the valve element 16. The limit element 18 is configured to make the valve element 16 rotate within a range of 0-90°, without exceeding 90°, thereby achieving the opening and closing of the valve element 16. That is to say, through the limit element 18, the valve element 16 is only able to achieve the opening and closing of the perfusion chamber 14 or the blood drawing chamber 13.

Valve elements 16 at the two ends of the diaphragm 15 are arranged in opposite directions to alternately achieve the opening and closing of the blood drawing chamber 13 and the perfusion chamber 14. For example, when the respiratory chamber 24 expands, the balloon 12 of the intracorporeal catheter 1 expands. At this point, the one-way valve located on the outer end side opens and the one-way valve located on the inner end side closes to close the perfusion chamber 14, thereby drawing blood from the body into the balloon 12. When the respiratory chamber 24 contracts, the balloon 12 of the intracorporeal catheter 1 contracts. At this point, the one-way valve located on the inner end side opens and the one-way valve located on the outer end side closes to close the blood drawing chamber, thereby perfusing blood from the balloon 12 into the body.

Specifically, the valve element 16 is made of a hard polymer material, a metal material, or a ceramic material. For example, the polymer material includes polyether ether ketone (PEEK), polyphenylsulphone (PPSU) or ultrahigh molecular weight polyethylene (UHMWPE). The metal material includes 316L stainless steel, cobalt chromium molybdenum alloy or titanium alloy. The ceramic material includes zirconia ceramic, alumina ceramic or zirconia toughened alumina ceramic. The valve element 16 and the rotating shaft 17 can be integrated or separated. When the valve element is integrated with the rotating shaft, the opening and closing of the valve element 16 can be achieved by the elastic deformation of the diaphragm 15 due to the elasticity of the diaphragm 15. However, this may result in the diaphragm 15 being prone to fatigue damage. Thus, the diaphragm 15 requires a relatively large thickness, in the range of 0.5-1.5 mm, and also requires a large toughness. For example, a more suitable material includes silicone rubber or PU. When the rotating shaft 17 and the valve element 16 are separated, a friction pair is formed between the valve element 16 and the rotating shaft 17, resulting in wear and debris between them. In order to minimize the friction and wear, a ceramic valve element and a ceramic rotating shaft are more suitable, which are made of zirconia ceramic, alumina ceramic, zirconia toughened alumina ceramic, etc.

In this embodiment, the wire 3 is configured to connect the respiratory chamber assembly 2 to the counterpulsation controller 4, providing electrical energy and a control signal to the respiratory chamber assembly 2, allowing the counterpulsation controller 4 to control the driver to perform a corresponding movement.

In this embodiment, the counterpulsation controller 4 adopts an IABP system controller to provide the electrical energy and control signal to the respiratory chamber assembly. The counterpulsation controller 4 has the following functions. 1) Waveform display function. Electrocardiograph (ECG), AP, BP waveforms can be displayed. For ECG, the piston action interval is adjustable. In addition, the catheter pressure can be accurately displayed. 2) Physiological data display function. Heart rate, assisted systolic/diastolic/mean/counterpulse pressure, unassisted systolic/diastolic/mean pressure can be displayed. 3) Icon display function. Battery capacity, piston status, and pressure value inside the respiratory chamber can be displayed. 4) The control methods include single touch screen control, button control, alarm corner control, and dual control of key/common functions. In touch screen/button mode, auxiliary startup, auxiliary frequency, screen freezing, printing, and reference line settings can be achieved. 5) The working modes include automatic and manual. The process of switching working modes does not affect normal counterpulsation. During the switching of working modes, the device automatically retains its original settings. In the automatic mode, the signal source, trigger mode (6 types), and phase algorithm are selected automatically; the ECG lead status is evaluated automatically, and the best ECG lead (7 types) is selected automatically. In the manual mode, the selection of the signal source and the trigger mode, the phase adjustment, and the selection of the ECG lead can be achieved. 6) 7 trigger modes are provided, including Pattern mode, Peak mode, Aifb mode, pacemaker V/A-V mode, pacemaker A mode, AP mode, and built-in setting mode. 7) Balloon delay analysis can be performed to calculate real-time balloon perfusion velocity and evaluate the safety of R-wave deflation. 8) There are four types of auxiliary frequencies, 1:1/1:2/1:4/1:8. 9) The maximum counterpulsation frequency can reach 200 beats per minute. 10) The counterpulsation capacity is 0-150 ml/time, which can be precisely adjusted with an accuracy of 0.5 mL. 11) Water can be automatically removed. When the driver does work, it generates heat, so cooling treatment is needed, but this produces condensed water. When the device is in use, it is necessary to remove condensed water every 20 minutes, automatically, without affecting normal assistance. 12) Regarding patient data reports, all patient information related to counterpulsation can be displayed and printed. 13) A power-on self-test checklist is provided, which displays self-test results in a list format. 14) Alarm history records of the last 100 alarms can displayed and printed.

Of course, the counterpulsation controller 4 can also adopt other structures as long as it can achieve functions similar to the IABP system controller.

In the present disclosure, the outside aortic counterpulsation device with an extracorporeal respiratory chamber operates on a different principle from IABP. It no longer uses any gas as a power source, but directly drives liquid motion. The driving process of the outside aortic counterpulsation device is completed outside the body, which is more controllable and safer. The balloon of the outside aortic counterpulsation device is placed outside the body, rather than inside the body like the IABP. Inside the body, the length and diameter of the aorta are limited. The IABP can provide up to three specifications: 30 cc, 40 cc, and 50 cc, with a maximum cardiac output of only 1 L/min. When the balloon is placed outside the body, the enclosed space formed by the intracorporeal catheter 1 inside the body can simulate the aorta, or in other words, this space is an extension of the human aorta. When the intracorporeal catheter 1 is set outside the body, its size, shape, and volume can be designed based on the maximum cardiac output. For example, the actual cardiac output that a 200 ml extracorporeal balloon can achieve may reach 5-10 L/min, completely solving the significant defect of insufficient cardiac output in IABP. Moreover, the overall device still retains all the advantages of the IABP.

The above are only preferred embodiments of the present disclosure, and are not intended to limit the present disclosure in any form. Simple alterations, equivalent changes or modifications made by those skilled in the art make using the technical contents disclosed above fall within the scope of protection of the present disclosure.

1. An outside aortic counterpulsation device with an extracorporeal respiratory chamber, comprising an intracorporeal catheter extending into a descending aorta and a respiratory chamber assembly located outside a body, wherein a first end of the respiratory chamber assembly is connected to an outer end of the intracorporeal catheter, and a second end of the respiratory chamber assembly is connected to a counterpulsation controller;
a side wall of an inner end of the intracorporeal catheter is provided with a plurality of through holes, and the outer end of the intracorporeal catheter is hermetically connected to an extracorporeal balloon; and
the respiratory chamber assembly comprises a housing, a piston driving mechanism and an elastic respiratory chamber membrane, wherein the piston driving mechanism and the elastic respiratory chamber membrane are arranged inside the housing; a closed end of the elastic respiratory chamber membrane is fixedly connected to a piston of the piston driving mechanism, and an open end of the elastic respiratory chamber membrane wraps around the extracorporeal balloon and is hermetically connected to an outer side of a base of the extracorporeal balloon; a respiratory chamber is formed between the elastic respiratory chamber membrane and the extracorporeal balloon; the piston driving mechanism is configured to drive the elastic respiratory chamber membrane to expand or contract; when the elastic respiratory chamber membrane expands, a vacuum is formed inside the respiratory chamber, allowing the extracorporeal balloon to be blown in an opposite direction, wherein blood from the descending aorta is drawn into the extracorporeal balloon; and when the elastic respiratory chamber membrane contracts, an internal space of the respiratory chamber is squeezed, allowing the blood in the extracorporeal balloon to be perfused into the body, thereby achieving outside aortic blood counterpulsation.

2. The outside aortic counterpulsation device with the extracorporeal respiratory chamber according to claim 1, wherein a lumen diameter of the intracorporeal catheter is 2.2 mm-9 mm; a hole setting section of the intracorporeal catheter has a length of 320 mm-520 mm; each of the plurality of through holes has a diameter of 1 mm-4 mm; and any two adjacent through holes of the plurality of through holes have a spacing of 1 mm-10 mm.

3. The outside aortic counterpulsation device with the extracorporeal respiratory chamber according to claim 1, wherein a diaphragm is provided in the intracorporeal catheter and configured to divide an internal space of the intracorporeal catheter into a blood drawing chamber and a perfusion chamber; and a first end of the diaphragm is located at a hole setting starting position of the intracorporeal catheter, and a second end of the diaphragm is located at the base of the extracorporeal balloon.

4. The outside aortic counterpulsation device with the extracorporeal respiratory chamber according to claim 3, wherein two sides of the diaphragm are respectively adhered to an inner side wall of the intracorporeal catheter; and the first end and the second end of the diaphragm are inclined, such that an inner end of the blood drawing chamber and an outer end of the perfusion chamber form a horn mouth-shaped structure.

5. The outside aortic counterpulsation device with the extracorporeal respiratory chamber according to claim 4, wherein the diaphragm is an elastic membrane.

6. The outside aortic counterpulsation device with the extracorporeal respiratory chamber according to claim 5, wherein the intracorporeal catheter is made of a polymer material of polytetrafluoroethylene (PTFE), polyurethane (PU), Pebax or polyethylene (PE) through a blow molding or extrusion process; the extracorporeal balloon and the elastic respiratory chamber membrane each are made of elastic silicone rubber or PU; and the elastic membrane is made of a medical polymer material of elastic silicone rubber, PU, PTFE, Pebax or PE, and the membrane has a thickness of 0.5 mm-1.5 mm.

7. The outside aortic counterpulsation device with the extracorporeal respiratory chamber according to claim 3, wherein each of the first end and the second end of the diaphragm is provided with a one-way valve; the one-way valve comprises a valve element and a rotating shaft; the rotating shaft is fixed to each of the first end and the second end of the diaphragm; a first end of the valve element is rotatably connected to the rotating shaft, and a second end of the valve element matches an inner side wall structure of the intracorporeal catheter; the first end of the valve element is provided with a limit element for limiting a rotation angle of the valve element; and valve elements at the first end and the second end of the diaphragm are arranged in opposite directions to alternately achieve opening and closing of the blood drawing chamber and the perfusion chamber.

8. The outside aortic counterpulsation device with the extracorporeal respiratory chamber according to claim 7, wherein the valve element is made of a hard polymer material, a metal material, or a ceramic material.

9. The outside aortic counterpulsation device with the extracorporeal respiratory chamber according to claim 1, wherein a pressure sensor is provided at an outer side of the inner end of the intracorporeal catheter.

10. The outside aortic counterpulsation device with the extracorporeal respiratory chamber according to claim 1, wherein the counterpulsation controller is an intra aortic balloon pump (IABP) system controller for providing electrical energy and a control signal to the respiratory chamber assembly.

11. The outside aortic counterpulsation device with the extracorporeal respiratory chamber according to claim 2, wherein a pressure sensor is provided at an outer side of the inner end of the intracorporeal catheter.

12. The outside aortic counterpulsation device with the extracorporeal respiratory chamber according to claim 3, wherein a pressure sensor is provided at an outer side of the inner end of the intracorporeal catheter.

13. The outside aortic counterpulsation device with the extracorporeal respiratory chamber according to claim 4, wherein a pressure sensor is provided at an outer side of the inner end of the intracorporeal catheter.

14. The outside aortic counterpulsation device with the extracorporeal respiratory chamber according to claim 5, wherein a pressure sensor is provided at an outer side of the inner end of the intracorporeal catheter.

15. The outside aortic counterpulsation device with the extracorporeal respiratory chamber according to claim 6, wherein a pressure sensor is provided at an outer side of the inner end of the intracorporeal catheter.

16. The outside aortic counterpulsation device with the extracorporeal respiratory chamber according to claim 7, wherein a pressure sensor is provided at an outer side of the inner end of the intracorporeal catheter.

17. The outside aortic counterpulsation device with the extracorporeal respiratory chamber according to claim 8, wherein a pressure sensor is provided at an outer side of the inner end of the intracorporeal catheter.
